(19) United States
(12) Patent Application Publication
(10) Pub. No.: US 2007/0249839 A1
(43) Pub. Date: Oct. 25, 2007

(54) Process for the Preparation of Losartan Potassium Form I

(76) Inventors: Bakulesh Mafatlal Khamar, Gujarat (IN); Indravadan Ambalal Modi, Gujarat (IN); Madhusudana Rao Gajula, Gujarat (IN); Radha Achanatha, Gujarat (IN); Murali Rajappa, Gujarat (IN)

Correspondence Address: Raj S Dave;DARBY & DARBY, Suite 250, 1500 K Street NW, Washington, DC, 20005-1714, US

(21) Appl. No.: 10/570,824

(22) PCT Filed:
(86) PCT No.: PCT/IB04/02879

(30) Foreign Application Priority Data
Sep. 4, 2003 (IN) 907/MUM/2003

(51) Int. Cl. C07D 257/04

(52) U.S. Cl. 548/107

(57) ABSTRACT

The present invention relates to a novel method for preparing trityl losartan and losartan potassium form I.

### FIELD OF THE INVENTION

This invention relates to a process for the preparation of losartan potassium and its polymorphic form I.

### BACKGROUND OF THE INVENTION

2-butyl-4-chloro-1-{[2′-(1H-tetrazol-5-yl) [1,1′-biphenyl]-4-yl]methyl}-1H-imidazole-5-methanol commonly known as Losartan, is useful in the treatment of hypertension as an AT, selective angiotensin II antagonist. Losartan is formulated as its potassium salt (I). 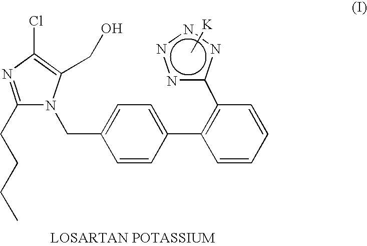
Losartan is generally prepared from 2-Butyl-4-chloro-5-(hydroxymethyl)-1-[[2′-[(triphenylmethyl)tetrazol-5-yl]biphenyl-4-yl]methyl]imidazole, commonly referred as Trityl Losartan (II). Several methods for the preparation of trityl losartan are known in literature and the following paragraphs briefly describe them.

EP 253310 discloses a process, wherein 2-n-butyl-4-chloro-1H-imidazolyl-5-methanol (III) is coupled with 5-(4′-bromomethyl-1,1′-biphenyl-2-yl)-2-triphenylmethyl-2H-tetrazole (IV) in N,N-dimethylformamide as solvent in presence of sodium methoxide as the base to furnish trityl losartan. The other bases that have been claimed are sodium hydride, alkali metal carbonates such as sodium carbonate and potassium carbonate and amine bases such as triethyl amine and pyridine.

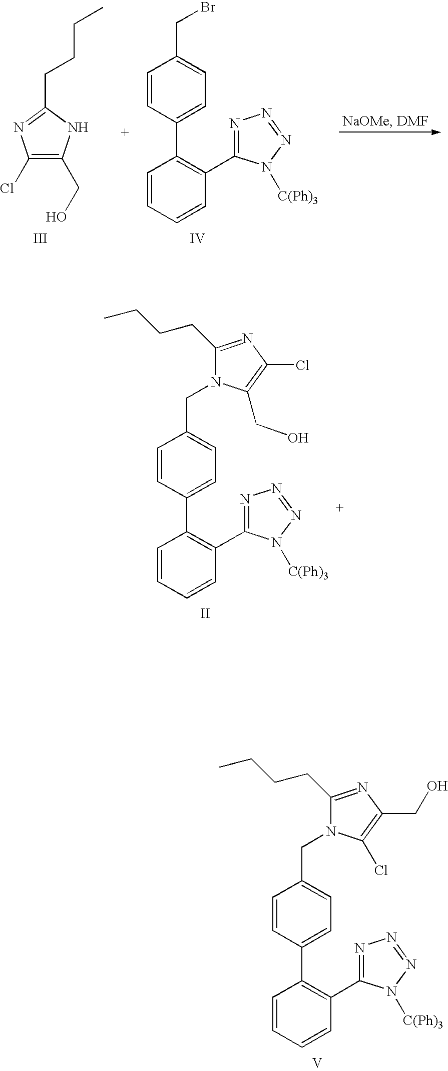

The coupling reaction results in a mixture of trityl losartan and its regio isomer (V). These are separated by column chromatography.

U.S. Pat. Nos. 5,130,439 and 5,310,928 disclose a method for coupling (IV) and (VI) in N,N-dimethylacetamide solvent in the presence of anhydrous potassium carbonate as base. The imidazole aldehyde (VI) gives predominantly the desired regio isomer (VII). The intermediate VII is then reduced with sodium borohydride to furnish the trityl losartan. The product is isolated by extraction into toluene from aqueous N,N-dimethylacetamide, concentration of the toluene solution and crystallization using ethyl acetate or ethanol as solvent. The synthesis steps are depicted as follows.

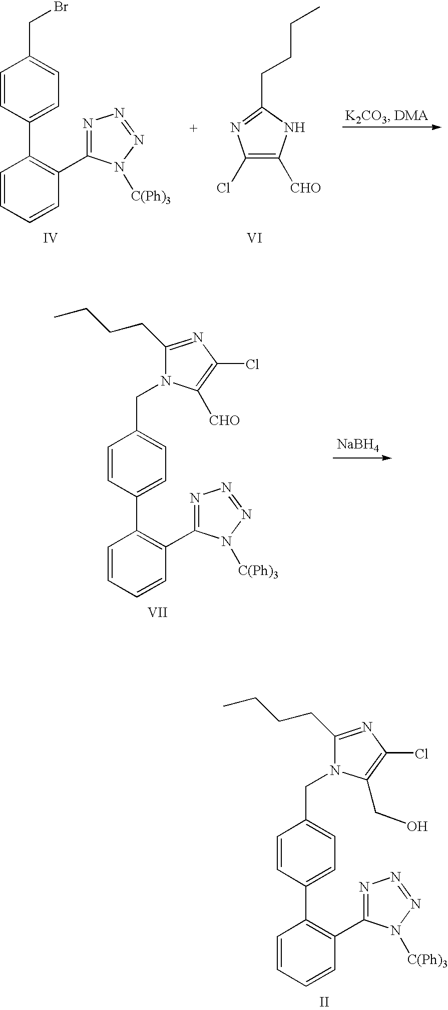

In a process published in J. Med. Chem. (1991), 34, 2525-2547, Losartan is prepared by coupling (III) and (IV) in N,N-dimethylformamide in the presence of sodium methoxide. The desired compound is isolated after vacuum distillation of solvent followed by extractive work-up. The resultant product mixture is purified by chronmatography.

The U.S. Pat. Nos. 5,138,069, 5,128,355 and 5,155,118 describe a process for the preparation of losartan, wherein the tetrazole ring of losartan is formed by reacting 1-((2′-cyanobiphenyl-4-yl)methyl)-2-butyl-4-chloro-5-hydroxymethylimidazole with trimethyltin azide. The reaction results in trimethylstannyl substituted tetrazole compound, which is then reacted with trityl chloride and sodium hydroxide.

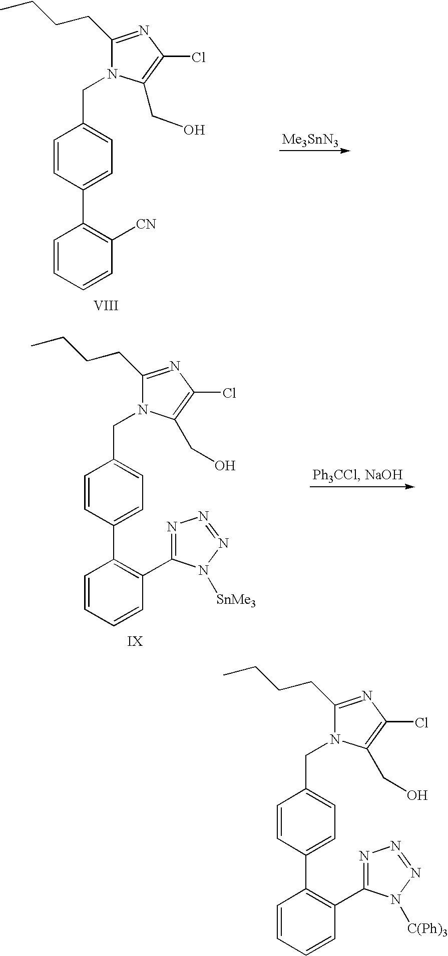

The trityl losartan thus formed is treated with 3.4N hydrochloric acid in methanol at about 10° C. to give losartan.

The U.S. Pat. Nos. 5,138,069, 5,128,355 and 5,155,118 also disclose another process for making trityl losartan, where in the coupling between IV and VI is carried out in a biphasic solvent system comprising of chlorinated solvent and water. The reaction is carried out at room temperature in presence of sodium hydroxide as the base and aliquat 336 as the phase transfer catalyst. The resulting intermediate VII is then reduced in situ with sodium borohydride to furnish trityl losartan.

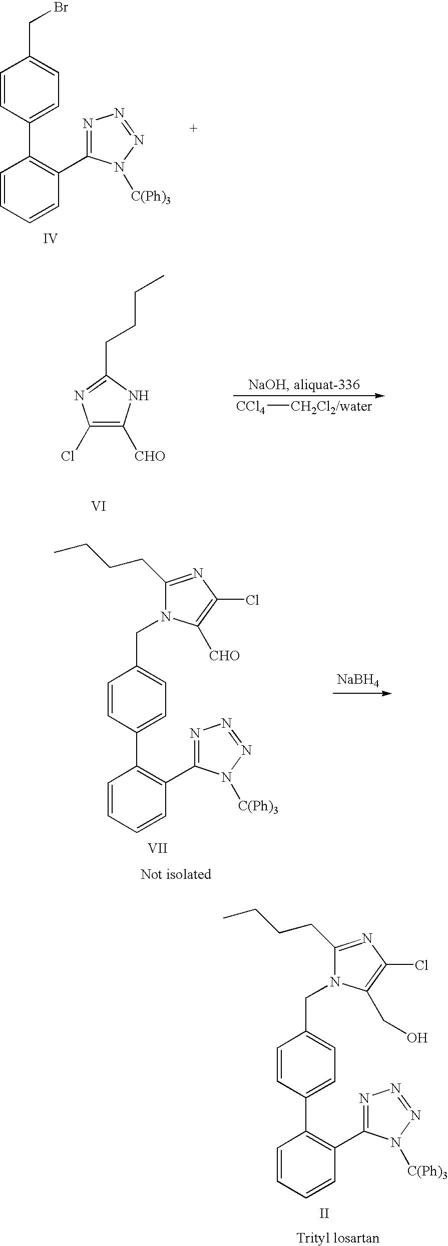

U.S. Pat. No. 5,206,374, 5,310,928 and 5,962,500 disclose another process for preparing losartan in which 5-phenyltetrazole (X) is converted into the boronic acid coupling partner (XII) for the Suzuki reaction by tritylation of phenyltetrazole with trityl chloride in presence of a non-nucleophilic base, ortho metalation with n-butyl lithium, followed by reaction with triisopropylborate. 2-n-butyl-4-chloro-1H-imidazole-5-carboxaldehyde (VI) is alkylated with 4-bromobenzylbromide, followed by reduction of the aldehyde with sodium borohydride to yield the other Suzuki coupling partner (XIII). The product of Suzuki coupling is trityl losartan. This process is published in J. Org. Chem. (1994), 59, 6391-6394.

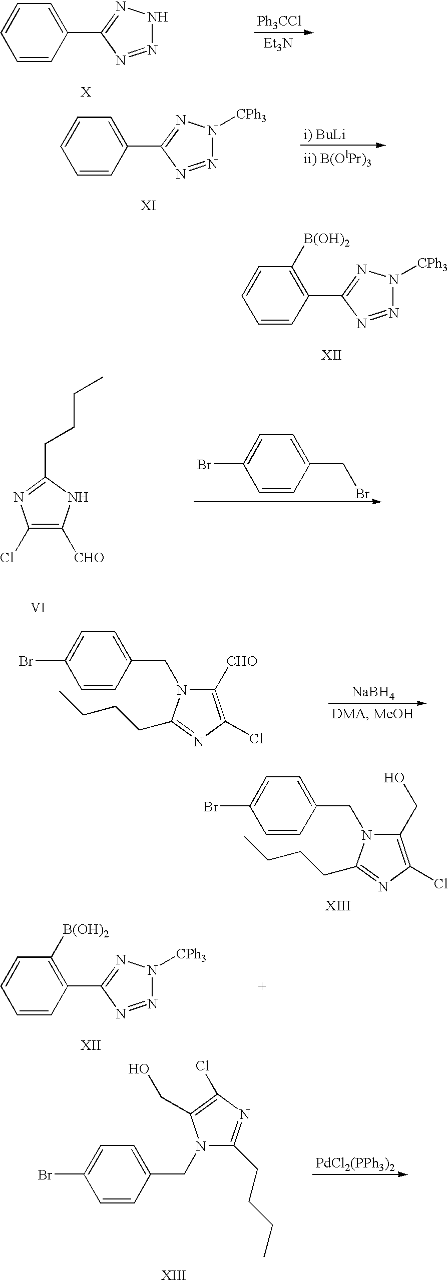

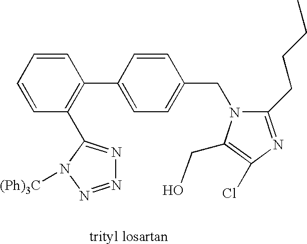

European patents EP 470,794 and EP 470,795 describe a method for the manufacture of biphenyl carbonitriles (XVI). These patents also describe a method of preparation of trityl losartan by coupling of intermediates (III) and (IV) employing the procedure described in EP 253,310.

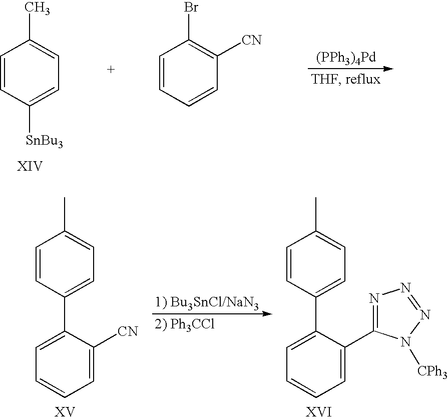

Losartan potassium exhibits polymorphism. Several polymorphic forms have been prepared and characterized. The following paragraphs briefly describe various polymorphs.

U.S. Pat. No. 5,608,075 discloses the polymorphic forms of losartan, wherein the trityl losartan is deprotected with H₂SO₄ in 50:50 acetonitrile:water and the free acid is treated with KOH solution. The aqueous solution containing losartan potassium is added slowly to a refluxing azeotropic mixture of cyclohexane/iso propanol and the ternary azeotrope cyclohexane/iso propanol/water is distilled till the water content of the pot is less than 0.05%. The white crystalline solid thus obtained is polymorphic form-I, which is characterized by DSC, XRD and IR. Polymorphic form-II is prepared by heating form-I in a DSC cell. This process is also described in U.S. Pat. No. 5,859,258.

U.S. Pat. No. 6,710,183 discloses the synthesis of losartan potassium starting from trityl losartan, wherein trityl losartan is reacted in an alcohol of formula R—OH (where R is C₁ to C₄ straight chain alkyl group) with 0.1 to 1 equivalent KOH. Losartan potassium thus formed is isolated after crystallizing out by changing the solvent to an aprotic or weakly protic solvent. The alcohol used is preferably methanol and the protic dipolar solvent used for the crystallization of the final product is preferably acetonitrile or straight or branched chain or cyclic aliphatic hydrocarbons.

EP 1294712 (WO 02/094816) discloses the process to manufacture losartan potassium form-I, wherein trityl losartan or losartan is suspended in a solvent and KOH is added to obtain a clear solution, which is then concentrated under reduced pressure to remove most of the solvent. An anti solvent is added to crystallize losartan potassium. The solvents to prepare losartan potassium include methanol, ethanol, and butanol but preferably the salt formation is carried out in methanol. Anti solvent is selected from common solvents such as ethyl acetate, acetonitrile, toluene and acetone, but the preferred anti solvent is acetone.

US application 2004/0006237 (WO 03/048135) relates to novel amorphous and novel crystalline forms III, IV, V of losartan potassium and the processes for their preparation. The patent also discloses novel processes for preparing losartan potassium forms I and II. The preparation of amorphous losartan includes the step of dissolving losartan potassium in a solvent to form a solution and distilling the solvent form the solution to dryness. Losartan form III (hydrated) is obtained by exposing losartan potassium amorphous or form I to an atmosphere having high relative humidity. Losartan potassium form IV is obtained by treating a saturated solution of losartan potassium in ethanol with methylene chloride. Losartan form V is obtained by treating a saturated solution of losartan potassium in ethanol with hexane. Losartan potassium form II is obtained by adding a saturated solution of losartan potassium in ethanol to xylene to form a mixture and evaporating ethanol from the mixture. Losartan form I is obtained by treating a saturated solution of losartan potassium in ethanol or iso propanol, with less soluble solvent like ethyl acetate, toluene, acetone, methyl ethyl ketone, methylene chloride, acetonitrile, dimethyl carbonate or hexane.

US application 2004/0034077 (WO 03/093262) discloses a process for preparing losartan and losartan potassium, wherein trityl losartan is treated with an acid in a diluent comprising a ketone. Especially preferred liquid ketones are acetone, methyl ethyl ketone and methyl isobutyl ketone, and acetone being the most preferred. Acids, which have been found suitable, include hydrochloric acid, sulphuric acid, acetic acid, trifluoroacetic acid, hydrobromic acid and formic acid. After the trityl losartan has been substantially converted to losartan, reaction mixture is basified. Preferred bases are alkali metal hydroxides and alkoxides. After addition of the base, the liquid ketone is evaporated under vacuum. After separation of triaryl methyl alcohol the residue is acidified to yield losartan. Free losartan is suspended in an alcohol and treated with a solution of potassium ions. Finally losartan potassium is precipitated from the alcohol. The alcohol is selected from the group consisting of isopropyl alcohol, butyl alcohol and isobutyl alcohol. The potassium ion solution is prepared by dissolving potassium iso propoxide, potassium butoxide and potassium iso butoxide or potassium hydroxide in the diluent.

US application 2004/0097568 discloses a process for preparing form III of losartan potassium, wherein trityl losartan is treated with aqueous solution of potassium hydroxide in methanol to obtain losartan potassium. The solvent is evaporated under vacuum and traces of water are removed as an azeotrope with toluene. Methanol and carbon are added to the resulting mixture. The carbon is filtered and the methanol is distilled. The resulting mixture is cooled to 20-25° C. to obtain crystalline form III losartan potassium.

The processes described above for the preparation of trityl losartan have several disadvantages. These include use of highly toxic and hazardous reagents like trialkyl tin azides, expensive and sensitive catalysts like tetrakis (triphenyl phosphine) palladium or highly reactive bases like butyl lithium. Some of the reactions are conducted at very low temperatures. Many of these procedures use water miscible solvents like N,N-dimethyl formamide or tetrahydrofuran. The reactions carried out in a biphasic solvent system comprising of chlorinated solvent and water in the presence of a phase transfer catalyst results in very low yields. The literature procedures for the detritylation of trityl losartan are generally carried out in presence of an acid or a base. The isolation of free losartan from the acid catalyzed reaction involves extensive work up. The free losartan obtained is then crystallized from mixture of solvents to obtain form I. Therefore there exists a need for an improved process that eliminates the disadvantages of the prior art process for the synthesis of losartan potassium.

### SUMMARY OF THE INVENTION

The present invention relates to the disclosure of an efficient and cost effective method of synthesis of losartan potassium comprising the preparation of trityl losartan by reacting N-triphenylmethyl-5-[4′-(bromomethyl)biphenyl-2-yl]tetrazole [IV] and 2-n-butyl 4-chloro 1H-imidazol 5-carboxaldehyde [VI] in a biphasic solvent system comprising water and an organic solvent under phase transfer catalysis in alkaline conditions, followed by in situ reduction using sodium borohydride. The trityl losartan thus obtained is suspended in an alcoholic solvent and heated to temperature of about 35 to reflux temperature of the solvent in the absence of acid or base catalysts to deprotect trityl losartan. The solvent is distilled off under reduced pressure to leave a residue that is then taken up in an organic solvent. The precipitated free losartan is filtered off and free losartan is suspended in an organic solvent capable of forming azeotrope with water and treated with aqueous potassium hydroxide solution. The solution is distilled to remove water as an azeotrope till moisture content of the mixture is less than 0.1%. Upon cooling losartan potassium crystallizes in polymorphic form-I and is isolated by filtration.

Alternatively, the residue obtained after distillation of alcoholic solvent is taken up in an organic solvent and treated with aqueous potassium hydroxide solution to dissolve losartan as its potassium salt. The layers are separated and water is removed from the aqueous solution as an azeotrope with an organic solvent as before.

### DETAILED DESCRIPTION OF THE INVENTION

The aim of the present invention is to develop an improved process that eliminates the disadvantages of the prior art process for synthesis of Trityl Losartan and also to provide a novel process to prepare polymorphic form I of Losartan potassium.

The present invention provides an improved process that eliminates the disadvantages of the prior art processes for synthesis of trityl losartan and also to eliminate extensive purification procedures to separate the regio isomer. According to the invention, the coupling reaction between intermediates (IV) and (VI) is carried out in presence of a base and phase transfer catalyst in an aqueous/organic biphasic solvent system where organic solvent is selected from toluene, xylene, pentane, heptane, octane, cyclohexane etc. Preferably the reaction is carried out in toluene. The reaction temperature varies from 25° C. to reflux temperature of the solvent, preferably from 80 to 100° C.

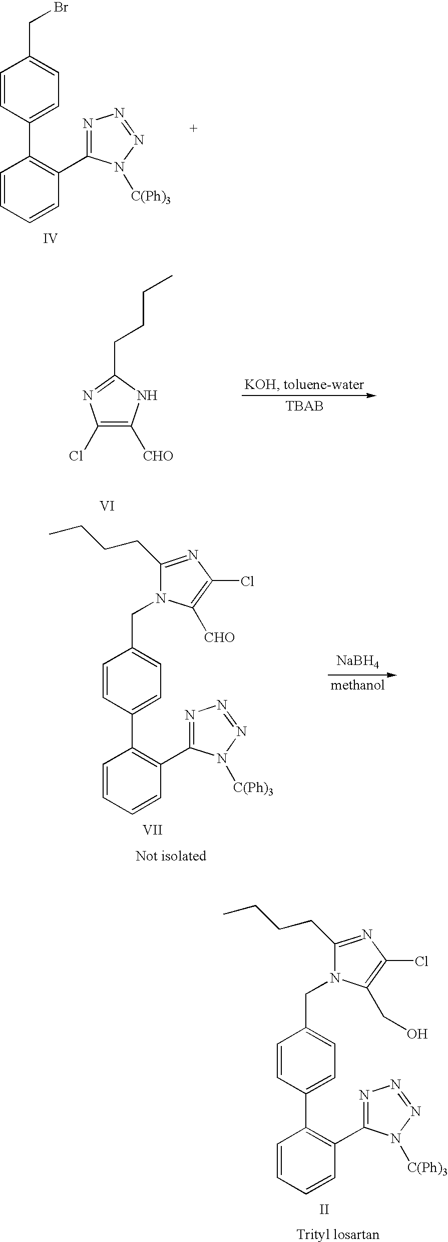

The phase transfer catalyst is selected from any of the tetra alkyl ammonium halides or tetra alkyl phosphonium halides. The preferred catalyst is tetra butyl ammonium bromide because of its easy availability and low cost. The quantity of the catalyst used varies from about 0.1 to 5 mol % with about 2 mol % being most suitable. The base employed is selected from alkali metal hydroxides such as lithium hydroxide; sodium hydroxide or potassium hydroxide the preferred base is potassium hydroxide in about 100 mol % to 300 mol % with about 130 mol % being most suitable. The reaction is carried out from ambient temperature to reflux temperature of the solvent and it generally takes 2-10 hours to go to completion. Typically, the reaction is conducted at 80-90° C. for about 3-4 hours. After the reaction has proceeded to a desired stage as judged by HPLC analysis, the reaction mixture is diluted with water and allowed to settle. The aqueous phase is removed and the organic phase is washed with water to remove any traces of phase transfer catalyst and base. The organic phase is then diluted with an alcohol selected from any of C1-C4 alcohols, preferably methanol and reduced with sodium borohydride in quantities ranging from 0.2 equivalents to 2 equivalents. Typically, about 0.5 equivalent of sodium borohydride is, used. The reduction is carried out in a temperature range of about −10 to +20° C. with 0-5° C. being most appropriate. The alcohol acts as a protonating source during the reduction of the aldehyde VII to trityl losartan. When the reaction has proceeded to a desired stage, it is quenched by addition of water. The desired product precipitates from the reaction mass. It is then isolated by filtration. The product thus isolated is about 96% pure and can be utilized for the production of Losartan potassium directly without any further purification. Thus in a “one pot” operation, synthesis of trityl losartan is achieved.

The present invention also provides novel methods for preparing losartan potassium form-I. According to the literature procedures, the detritylation is generally carried out in presence of an acid or a base. The cleavage of the trityl group is achieved with strong acid such as hydrochloric acid or sulphuric acid and the desired losartan potassium is isolated after extensive work up. In case of the base catalyzed reaction, trityl losartan is treated with potassium hydroxide in methanol to obtain losartan potassium in situ.

Surprisingly we found that losartan can be easily obtained by heating trityl losartan in an alcoholic solvent, in the absence of an acid or a base catalyst. The alcoholic solvent is selected from any C1-C4 alcohols. The preferred solvent is methanol. The reaction is carried out at ambient temperature to reflux temperature of the solvent and it generally takes 2-12 hour for completion. Typically the reaction is conducted at reflux temperature for 4-5 hour. After completion of the reaction, alcoholic solvent is distilled off under reduced pressure. The resulting mass containing trityl methyl ether (by product) and free losartan is suspended in an organic solvent (A). The organic solvent (A) is selected from the group consisting of toluene, ethyl acetate, acetone, acetonitrile and methylene chloride. The preferred solvent is toluene. The trityl methyl ether being freely soluble in toluene is extracted into solvent where as the free losartan remains insoluble. The resulting suspension is filtered and washed with toluene to obtain free losartan. The free losartan obtained is suspended in another organic solvent (B) that is capable of forming an azeotrope with water and treated with aqueous potassium hydroxide solution to obtain a clear solution. The quantity of base used varies from about 0.98-1 equivalent with respect to the starting material, with 1 equivalent being most suitable.

The solution is distilled to remove water as an azeotrope till moisture content of the mixture is less than 0.1%. Upon cooling losartan potassium crystallizes in polymorphic form-I and is isolated by filtration.

Thus a suspension of trityl losartan in methanol is refluxed for about 4-5 h. The solid slowly dissolves and deprotection takes place. After completion of the reaction, methanol is distilled off under reduced pressure. The resulting mass is taken up in toluene in which trityl methyl ether is freely soluble. The free losartan remains insoluble and is filtered. The free losartan thus obtained is suspended in a solvent capable of forming an azeotrope with water and treated with concentrated solution of aqueous potassium hydroxide. The mixture is stirred to obtain a clear solution. The solvents can be selected from acetonitrile, acetone, 2-butanone, ethyl acetate and toluene. The preferred solvent is acetonitrile. The solution is distilled to remove water as an azeotrope (boiling point 76.5° C.). The distillation is continued till the moisture content of the solution is less than 0.1%. The mass is cooled to room temperature and crystalline solid thus obtained is filtered and found to be form I as established by extensive analytical methods.

In another embodiment of this invention, the free losartan is suspended in acetonitrile and treated with concentrated solution of aqueous potassium hydroxide. The mixture is stirred to obtain a clear solution. The solution is diluted with isopropyl ether and the resulting solution is distilled to remove water as tertiary azeotrope (59° C.). The ternary azeotrope has a lower boiling point than the binary azeotrope. The distillation is continued till the moisture content of the solution is less than 0.1%. The crystalline solid thus obtained is filtered and found to be form I as established by extensive analytical methods.

In another embodiment of this invention, a suspension of trityl losartan in methanol is refluxed for about 4-5 hours. After completion of the reaction, methanol is distilled off under reduced pressure. The resulting mass is suspended in toluene and treated with aqueous potassium hydroxide solution. The mixture is stirred to obtain a clear solution. The aqueous layer containing losartan potassium is separated. Fresh toluene is added and water is removed as an azeotrope with toluene (85° C.). The distillation is continued till the moisture content of the solution is less than 0.1%. The crystalline solid thus obtained is filtered and found to be form I.

In another embodiment of this invention, trityl losartan is treated with ethanolic potassium hydroxide solution. The mixture is diluted with water to precipitate trityl methyl ether. The precipitated trityl methyl ether is filtered. Resulting solution is further diluted with 2-butanone. The solution is distilled to remove water as a ternary azeotrope (73.2° C.). When the solution is dry the potassium salt crystallizes. The crystalline solid thus obtained is filtered and found to be form I as established by extensive analytical methods.

The present invention is illustrated with following examples without limiting, the scope of the invention.

### EXAMPLE 1

Preparation of Trityl Losartan:

To a solution of N-(triphenylmethyl)-5-[4′-(bromomethyl)biphenyl-2-yl]tetrazole [1.0 Kg; 1.61 mol], 2-n-butyl 4-chloro 1H-imidazol 5-carboxaldehyde [0.28 kg; 1.501 mol] and tetra butyl ammonium bromide [0.01 kg; 0.03 mol] in 5L toluene is added 0.6L of a 3M solution of potassium hydroxide in water. The reaction is vigorously stirred and refluxed for 3.5 hour. The progress of the reaction is monitored by HPLC. The reaction mass is cooled to room temperature and is allowed to settle. The lower aqueous layer is discarded and the toluene layer is washed with water (2L). The toluene layer is diluted with methanol (0.6 L) and cooled to 0° C. Sodium borohydride (0.031 kg, 0.837 mol) is added in lots maintaining the temperature below 5° C. Progress-of the reaction is monitored by HPLC. The reaction is stopped by addition of 3L water.

The precipitated solid is filtered, washed with toluene (0.5 L) and dried to give Trityl losartan

Yield: 0.9 kg. [90.2%]

¹H NMR (CDCl₃): δ 9.73(s, 1H); 7.92 (m, 1H); 7.51-6.81 (m, 2H); 5.45(s, 2H); 2.49(t, 2H); 1.64(q, 2H); 1.28 (sextet, 2H) and 0.86 (t, 3H)

### EXAMPLE 2

Preparation of Trityl Losartan:

To 50 ml of xylene, N-(triphenylmethyl)-5-[4′-(bromomethyl)biphenyl-2-yl]tetrazole [9.0 g; 0.0164 mol], 2-n-butyl 4-chloro 1H-imidazol 5-carboxaldehyde [2.83 g; 0.015 mol], tetra butyl ammonium bromide [0.217 g; 0.0006 mol] and a solution of potassium hydroxide (1.21 g in 15 ml of water) are added. The reaction is vigorously stirred and refluxed for 3.5 hours. The progress of the reaction is monitored by HPLC. The reaction mass is cooled to room temperature and is allowed to settle. The lower aqueous layer is discarded and the xylene layer is washed with water (50 ml). Further it is diluted with methanol (20 ml) and cooled to 0° C. Sodium borohydride (0.3367 g, 0.0089 mol) is added in lots maintaining the temperature below 5° C. Progress of the reaction is monitored by HPLC. The reaction is stopped by addition of water (30 ml). The precipitated solid is filtered, washed with xylene (10 ml) and dried.

Yield: 8.8 g [74.01%].

### EXAMPLE 3

Preparation of Trityl Losartan:

To 50 ml of toluene, N-(triphenylmethyl)-5-[4′-(bromomethyl)biphenyl-2-yl]tetrazole [9.0 g; 0.0164 mol], 2-n-butyl 4-chloro 1H-imidazol 5-carboxaldehyde [2.83 g; 0.015 mol], tetra butyl ammonium bromide [0.217 g; 0.0006 mol] and a solution of potassium carbonate (3.1 g in 15 ml of water) are added. The reaction is vigorously stirred and refluxed for 3.5 hours. The progress of the reaction is monitored by HPLC. The reaction mass is cooled to room temperature and is allowed to settle. The lower aqueous layer is discarded and the toluene layer is washed with water (50 ml). Further it is diluted with methanol (20 ml) and cooled to 0° C. Sodium borohydride (0.3367 g, 0.0089 mol) is added in lots maintaining the temperature below 5° C. Progress of the reaction is monitored by HPLC. The reaction is stopped by addition of water (30 ml). The precipitated solid is filtered, washed with toluene (10 ml) and dried.

Yield: 8.0 g [66%]

### EXAMPLE 4

Preparation of trityl losartan:

To 50 ml of toluene, N-(triphenylmethyl)-5-[4′-(bromomethyl)biphenyl-2-yl]tetrazole [9.0 g; 0.0164 mol], 2-n-butyl 4-chloro 1H-imidazol 5-carboxaldehyde [2.83 g; 0.015 mol], Aliquat-336 [0.14 g; 0.00035 mol] and a solution of potassium hydroxide (1.25 g in 15 ml of water) are added. The reaction is vigorously stirred and refluxed for 3.5 hours. The progress of the reaction is monitored by HPLC. The reaction mass is cooled to room temperature and is allowed to settle. The lower aqueous layer is discarded and the toluene layer is washed with water (50 ml). Further it is diluted with methanol (20 ml) and cooled to 0° C. Sodium borohydride (0.31 g, 0.0081 mol) is added in lots maintaining the temperature below 5° C. Progress of the reaction is monitored by HPLC. The reaction is stopped by addition of water (30 ml). The precipitated solid is filtered, washed with toluene (10 ml) and dried.

Yield: 6.7 g [62%].

### EXAMPLE 5

Preparation of trityl losartan:

To 50 ml of toluene, N-(triphenylmethyl)-5-[4′-(bromomethyl)biphenyl-2-yl]tetrazole [9.0 g; 0.0164 mol], 2-n-butyl 4-chloro 1H-imidazol 5-carboxaldehyde [2.83 g; 0.015 mol], cetrimide [0.03 g; 0.00035 mol] and a solution of potassium hydroxide (1.29 g in 1.5 ml of water) are added. The reaction is vigorously stirred and refluxed for 3.5 hours. The progress of the reaction is monitored by HPLC. The reaction mass is cooled to room temperature and is allowed to settle. The lower aqueous layer is discarded and the toluene layer is washed with water (50 ml). Further it is diluted with methanol (20 ml) and cooled to 0° C. Sodium borohydride (0.311 g, 0.0082 mol) is added in lots maintaining the temperature below 5° C. Progress of the reaction is monitored by HPLC. The reaction is stopped by addition of water (30 ml). The precipitated solid is filtered, washed with toluene (10 ml) and dried.

Yield: 7.9 g [68%].

### EXAMPLE 6

Preparation of trityl losartan:

To 50 ml of methylene chloride, N-(triphenylmethyl)-5-[4′-(bromomethyl)biphenyl-2-yl]tetrazole [10.2 g; 0.018 mol], 2-n-butyl 4-chloro 1H-imidazol 5-carboxaldehyde [3.4 g; 0.018 mol], tetra butyl ammonium bromide [0.2618 g; 0.00081 mol] and a solution of potassium hydroxide (1.45 g in 15 ml of water) are added. The reaction is vigorously stirred and refluxed for 12 hours. The progress of the reaction is monitored by HPLC. The reaction mass is cooled to room temperature and is allowed to settle. The upper aqueous layer is discarded and the methylene chloride layer is washed with water (50 ml). Further it is diluted with methanol (20 ml) and cooled to 0° C. Sodium borohydride ((1.3367 g, 0.009 mol) is added in lots maintaining the temperature below 5° C. Progress of the reaction is monitored by HPLC.

The solvent is removed under reduced pressure and the residue obtained is dissolved in toluene 30 ml. Distillation is continued until the temperature reaches 110° C. The reaction mass is cooled to 40° C. and then diluted with 15 ml of ethyl acetate and 25 ml of n-heptane. The reaction mixture is further cooled to 0-10° C. and stirred for about 10 minutes. The slurry obtained is filtered. Washed with cold toluene/ethyl acetate.

Yield: 7.5 g [62%].

### EXAMPLE 7

Preparation of Free Losartan:

Trityl losartan (50 g; 0.075 mol) is suspended in methanol (250 ml). The resulting mixture is refluxed for 4-5 hours to obtain a clear solution. Methanol is distilled off under reduced pressure. The resulting mass is suspended in toluene (250 ml) and stirred at 45-50° C. for about 15 min. The free losartan, which is insoluble, is filtered and washed with toluene.

Yield: 28g (88%)

### EXAMPLE 8

Preparation of Losartan Potassium Form I:

Trityl losartan (200 g; 0.3 mol) is suspended in methanol (1L). The resulting mixture is refluxed for 4-5 hours to obtain a clear solution. Methanol is distilled off under reduced pressure. The resulting mass is suspended in toluene (1 L) and treated with a solution of potassium hydroxide (85%, 20 g; 0.3 mol) in water (500 ml). The mixture is stirred for 15-20 min. to obtain a clear solution. The aqueous layer containing losartan potassium is separated. Fresh toluene (1.2L) is added to the aqueous solution. The resulting mixture is distilled to remove water as an azeotrope. The distillation is continued till the moisture content of the solution is less than 0.1%. The crystalline solid thus obtained is filtered and found to be form I as established by extensive analytical methods.

Yield: 125 g (92%)

### EXAMPLE 9

Preparation of Losartan Potassium Form I:

A suspension of free losartan (25 g; 0.059 mmol) in acetonitrile (150 ml) is treated with a solution of potassium hydroxide (85%, 3.9 g; 0.059 mol) in water (20 ml). The mixture is stirred for 15-20 min. to obtain a clear solution. The solution is diluted with isopropyl ether (150 ml). The resulting solution is distilled to remove water as an azentrope. The distillation is continued till the moisture content of the solution is less than 0.1%. The crystalline solid thus obtained is filtered and found to be form I as established by extensive analytical methods.

Yield: 25 g (92%)

### EXAMPLE 10

Preparation of Losartan Potassium Form I:

A suspension of free losartan (25 g; 0.059 mol) in acetonitrile (500 ml) is treated with a solution of potassium hydroxide (85%, 3.9 g; 0.059 mol) in water (15 ml). The mixture is stirred for 1.5-20 min. to obtain a clear solution. The resulting solution is distilled to remove water as an azeotrope. The distillation is continued with drop wise addition of acetonitrile (500 ml), till the moisture content of the solution is less than 0.1%. The crystalline solid thus obtained is filtered and found to be form I as established by extensive analytical methods.

Yield: 25 g (92%)

### EXAMPLE 11

Preparation of Losartan Potassium Form I:

A suspension of trityl losartan (10 g; 0.045 mol) in ethanol (60 ml) is treated with potassium hydroxide (85%, 1 g; 0.015 mol). Tlhe resulting mixture is refluxed for 24 hours. Cooled the mixture to room temperature and diluted with water (5 ml). The precipitated trityl methyl ether is filtered. The filtrate containing losartan potassium is further diluted with methyl ethyl ketone (150 ml). The resulting solution is distilled to remove water as an azeotrope. When the solution is dry the potassium salt crystallizes. The crystalline solid thus obtained is filtered and found to be form I as established by extensive analytical methods.

Yield: 5.2 g (82%)

[image]

| Angle | d value | Intensity % |
|---|---|---|
| 2-Theta° | Angstrom | % |
| 7.441 | 11.87168 | 70.7 |
| 11.227 | 7.87464 | 100.0 |
| 14.009 | 6.31668 | 2.5 |
| 14.377 | 6.15595 | 36.7 |
| 14.922 | 5.93230 | 10.3 |
| 15.251 | 5.80502 | 5.2 |
| 16.060 | 5.31433 | 7.5 |
| 17.314 | 5.11750 | 10.3 |
| 18.688 | 4.74445 | 7.6 |
| 19.106 | 4.64139 | 11.0 |
| 19.368 | 4.57935 | 4.3 |
| 20.150 | 4.40340 | 2.7 |
| 21.015 | 4.22402 | 3.2 |
| 21.548 | 4.12066 | 3.8 |
| 22.455 | 3.95618 | 25.7 |
| 22.877 | 3.88421 | 2.4 |
| 24.096 | 3.69046 | 24.8 |
| 24.688 | 3.61044 | 0.9 |
| 24.964 | 3.56402 | 5.7 |
| 26.371 | 3.37701 | 3.6 |
| 26.783 | 3.32600 | 7.2 |
| 27.542 | 3.23596 | 7.2 |
| 28.884 | 3.08858 | 14.2 |
| 29.353 | 3.04036 | 3.1 |
| 29.846 | 2.99119 | 19.9 |
| 31.140 | 2.86983 | 17.6 |
| 31.775 | 2.81392 | 2.2 |
| 33.308 | 2.68778 | 4.7 |
| 33.936 | 2.63951 | 4.5 |
| 35.106 | 2.55413 | 2.9 |
| 35.610 | 2.51917 | 4.0 |
| 36.224 | 2.47787 | 2.1 |
| 40.332 | 2.23445 | 1.9 |
| 43.724 | 2.06862 | 2.0 |
| 46.401 | 1.95531 | 2.0 |
| XCAD-06-85 | LOSARTAN K 081 AR02 | Apr. 2, 2004 |

[image]

We claim:

1) A process for preparing of losartan potassium comprising
a) Reacting 2-n-butyl-4-chloro 1H-imidazole 5-carboxaldehyde with N-(triphenylmethyl)-5-[4′-(bromomethyl)biphenyl-2-yl)]tetrazole in a biphasic solvent system comprising water and an organic solvent in the presence of a base and a phase transfer catalyst at ambient temperature to reflux temperature of the solvent for sufficient time to form an intermediate;
b) Separating the layers and diluting the organic layer containing the intermediate with an alcoholic co-solvent and reducing with sodium borohydride at −10 to 20° C. for 2 hours;
c) Diluting the reaction mixture with water to precipitate trityl losartan;
d) Stirring the trityl losartan in an alcoholic solvent in the absence of acid or base catalysis at ambient temperature to reflux temperature of the solvent for sufficient time to effect deprotection;
e) Distillation of the alcoholic solvent under reduced pressure to give a residue, which is mixed with an organic solvent (A) to precipitate losartan;
f) Suspending losartan in an organic solvent (B) capable of forming an azeotrope with water and treating with aqueous potassium hydroxide solution to provide a solution, to which another organic solvent (C) may optionally be added;
g) Distilling the solution to remove water as an azeotrope, till water content in the mixture is reduced below 0.1% to allow crystallization of losartan potassium in polymorphic form I;
h) Cooling the mixture to ambient temperature and isolating losartan potassium polymorph I by filtration.

2) The process of claim 1, wherein the alcoholic co-solvent used is selected from any of C1-C4 alcohols.

3) The process of claim 1, wherein C1-C4 alcohol solvent is selected from methanol, ethanol, propanol, isopropanol, n-butanol, iso butanol and tert-butanol

4) Process of claim 2, wherein the alcohol used is methanol.

5) Process of claim 2, wherein the alcohol used is ethanol.

6) The process of claim 1, wherein the reflux temperature of the solvent is maintained for 4-6 hours.

7) The process of claim 1, wherein the organic solvent of the biphasic solvent system in step a) is selected from the hydrocarbon solvents comprising of toluene, xylene, pentane, octane, cyclohexane and like.

8) The process according to claim 7, wherein the preferred organic solvent is toluene.

9) Process of claim 1 wherein the phase transfer catalyst in step a) is selected from the group of tetra alkyl ammonium halides other than aliquat-336 or tetra alkyl phosphonium halides excluding tetra butyl phosphonium bromide.

10) The process according to claim 9, wherein the preferred phase transfer catalyst is tetra butyl ammonium bromide.

11) The process of claim 1, wherein the base used in step a) is selected from alkali metal hydroxides.

12) The process of claim 1, wherein the base used in step a) is selected from alkali metal carbonates.

13) Process of claim 1, wherein the organic solvent (A) is selected from toluene, ethyl acetate, acetonitrile, acetone, 2-butanone, dichloromethane, isopropyl ether and like.

14) A process of claim 1, wherein addition of organic solvent (A) precipitates free losartan.

15) The process of claim 1, wherein losartan potassium is isolated from the solution by distillation of water with one or more solvents, which are capable of forming azeotrope with water.

16) Process of claim 1, wherein the organic solvent B capable of forming azeotrope is selected from acetonitrile, ethyl acetate, acetone, 2-butanone, toluene and like.

17) The process of claim 1, wherein the 0.98-1 equivalent of potassium hydroxide is used to obtain losartan potassium.

18) Process of claim 1, wherein the organic solvent C is selected from isopropyl ether, ethanol and like.

19) Process of claim 1, wherein the ternary azeotrope system obtained is acetonitrile-isopropyl ether-water when acetonitrile is used as solvent B and isopropyl ether is used as solvent C.

20) Process of claim 1 step F), wherein the ternary azeotrope system obtained when 2-butanone is used a solvent B and ethanol is used as solvent C, is 2-butanone-ethanol-water.

21) A process of claim 1, wherein the suspension obtained in step d) is treated with aqueous potassium hydroxide solution to obtain a two-phase liquid containing losartan potassium in water and trityl byproduct in organic solvent.

22) A process of claims 1 and 21, wherein the by-product is separated by extracting into organic solvent that is capable of dissolving, trityl by product but immiscible with water.

23) A process according to claim 22, wherein the organic solvent used is aromatic solvents such as toluene, halogenated solvents such as dichloromethane and ethereal solvents such as isopropyl ether and ester solvents such as ethyl acetate and like.

24) A process according to claim 1, wherein one or more organic solvents capable of forming azeotrope with water is added to the solution of losartan potassium in water.

25) A process according to claims 1 and 24, wherein liquid mixture is distilled to remove water as an azeotrope till water content in the mixture is less than 0.1% to allow crystallization of losartan potassium in polymorphic form I.

26) A process according to claim 25, wherein the organic solvent is selected from acetonitrile, ethyl acetate, isopropyl ether and toluene.

27) A process according to claim 1, wherein distillation in step g) is continued till water content in the mixture is less than 0.1% to allow crystallization of losartan potassium in polymorphic form I.
